(19) United States
(12) Patent Application Publication
(10) Pub. No.: US 2016/0354590 A1
(43) Pub. Date: Dec. 8, 2016

(54) MICRO NEEDLE STAMP

(71) Applicant: LAON CO., LTD., Siheung-si (KR)

(72) Inventor: Doo Won LEE, Ansan-si (KR)

(21) Appl. No.: 15/108,926

(22) PCT Filed: Dec. 23, 2014
(86) PCT No.: PCT/KR2014/012678

§ 371 Date: Jun. 29, 2016

(30) Foreign Application Priority Data
Dec. 31, 2013 (KR) 10-2013-0168394

(51) Int. Cl. A61M 37/00

(52) U.S. Cl. CPC A61M 37/0015; A61M 2037/0023

(57) ABSTRACT

A micro needle stamp comprises: a micro needle unit having a plurality of micro needles implanted therein; a main cover receiving and slidably supporting the micro needle unit; an auxiliary cover in which a plurality of openings are formed to correspond to the plurality of micro needles, and which is slidably coupled to the outer surface of the main cover while restricting the main cover from moving backward with respect thereto; a first resilient body interposed between the micro needle unit and the main cover; and a second resilient body interposed between the main cover and the auxiliary cover.

### TECHNICAL FIELD

The present invention relates to a micro needle stamp and more specifically to a micro needle stamp that stimulates skin by perforating an opening on the skin or be capable of being injected medication or the like through the opening perforated in the skin.

### BACKGROUND ART

Typically, a method that applies or sprays cosmetics or therapeutic agents in which particular components is contained on the skin is used so as to prevent wrinkles from generating on the skin or so as to treat skin troubles. However, in a case of this method, effect thereof is not great in fact since the particular components supplied on the skin are difficult to pass through stratum corneum of the skin.

In order to solve the problem described above, a method that uses a device typically referred to as “a micro needle” is introduced. The micro needle means a device that stimulates the skin using a small size of a needle, perforates a small opening on the skin and supplies nutrients required for collagen formation in a dermal layer, is capable of easily injecting the medication, or the like.

Also, a large molecular weight medication such as insulin, hormones is capable of being easily transmitted through the skin by the micro needle. When the micro needle penetrates into the dermal layer to stimulate the dermal layer, burns or scars can be treated naturally, and collagen generation is induced and thus skin tone can be improved and anti-aging effect can be expected. There are a roller type in which a plurality of the needles are planted in the roller and is rotated and a stamp type in which a plurality of the needles are planted in a circular or rectangular plate and is vibrated in the forward and backward direction as types of the micro needle. Hereinafter, the stamp type of the micro needle associated with the present invention will be described.

As an example of the stamp type of micro needle, a micro needle stamp that includes a stamp that has a plate shape and has a plurality of coupling openings, and a plurality of micro needles that are inserted into and fixed to the plurality of the coupling openings through one surface side of the stamp and has a cutting edge portion that projects from the other surface of the stamp by a predetermined length is disclosed in the Korea Utility Model Registration No. 20-0468393.

However, in a case of the micro needle stamp in the related art described above, the micro needle is always maintained with a projected state. Accordingly, in a case where the micro needle performs a stamping operation on one portion of the skin and then moves to other portion of the skin treatment, skin damage such as tearing is generated by movement of the micro needle in the lateral direction.

Meanwhile, in order to prevent skin from being damaged as above, the micro needle has to be move in the lateral direction with a sufficient gap between the skin and the micro needle and thus the skin is unlikely to be continuously treated. Because of this, workability is lowered.

### DISCLOSURE

### Technical Problem

The present invention has been devised to solve the problems as described above and an objective of the present invention is to provide a micro needle stamp that has an improved structure in order to fundamentally eliminate the skin damage resulting from the movement of the micro needle in the lateral direction and to be capable of continuously performing the treatment and thus to significantly improve the workability.

### Technical Solution

A micro needle stamp according to the present invention for achieving the above object, includes a micro needle unit in which a plurality of micro needles are planted; a main cover in which the micro needle unit is received and which supports to be slidable the micro needle unit; an auxiliary cover on which a plurality of openings are formed to correspond to the plurality of the micro needles, which restricts the main cover from relatively moving in the backward direction and couples with an outer surface of the main cover in a slidable manner; a first resilient body that is interposed between the micro needle unit and the main cover; and a second resilient body that is interposed between the main cover and the auxiliary cover.

The micro needle unit may include the plurality of the micro needles; a needle support body in which the plurality of the micro needles are planted to be exposed in the forward direction; a press block that detachably couples to a backward surface of the needle support body; and a press portion that connected to a backward surface of the press block. The needle support body may include a body in which the plurality micro needles are planted; one projection or a plurality of projections projected from a outer surface of the body, and the press block including a block that is in contact with a bottom surface of the body; and a coupling portion which extends from the block to receive the bottom portion of the body and has a projection groove penetrated corresponding to the projection.

The main cover may include a receiving portion that has a receiving space receiving the needle support body and the press block; a support portion that slidably supports the press portion; a connection portion that integrally connects the receiving portion with the support portion; a extension portion that integrally extends from the connection portion to be apart from a outer surface of the receiving portion, and a expansion portion that expands from the support portion to have a separation space between the press portion and the support portion. The first resilient body is preferably interposed between the press portion and the expansion portion.

The auxiliary cover may include a front surface portion that is spaced in front of the needle support body and has a plurality of openings corresponding to the plurality of the micro needles; a first sliding portion that extends from the front surface portion and is slidably in face-contact with a outer surface of the receiving portion; a second sliding portion that integrally extends to be expanded from the first sliding portion and is spaced from the outer surface of the receiving portion to the outside and is slidably in face-contact with an inner surface of the extension portion; and restricting means that restricts the extension portion from moving in the backward direction. The restricting means is one projection portion or a plurality of projection portions that extend from the second sliding portion to the backward side and has a retaining projection at the end thereof. At this time, a retaining groove corresponding to the retaining projection is penetrated through the extension portion. Further, the second resilient body is preferably interposed between the extension portion and the second sliding portion.

### Advantageous Effects

According to the micro needle stamp of the present invention as described above, when the micro needle moves from one portion of the skin to another portion of the skin in the lateral direction, a user releases the forward movement of the main cover, and thus the auxiliary cover returns to the original position by the resilient restoring force of the second resilient body. Accordingly, the micro needle is introduced into the secondary cover and prevents the skin from being damaged.

In particular, since the user can perform the treatment by moving the main cover in the forward direction while moving the micro needle in the lateral direction,

Further, since the needle support body in which the micro needle is implanted is detachably coupled to the press block, in a case where the micro needle is necessary to be replaced, only the needle support body in which the micro needle is implanted can be replaced and thus the inconvenience that replaces the entire micro needle unit is eliminated and the cost according to the replacement is reduced.

### DESCRIPTION OF DRAWINGS

FIG. 1 is a perspective view illustrating a micro needle stamp according to an embodiment of the invention,

FIG. 2 is a sectional perspective view illustrating a micro needle stamp in FIG. 1,

FIG. 3 is a sectional perspective view illustrating a state in which a main cover of the needle stamp in FIG. 1 is slid in the forward direction,

FIG. 4 is a sectional perspective view illustrating a state in which the micro needle is withdrawn and skin is treated in a state where the micro needle stamp in FIG. 3 is moved in the forward direction,

FIG. 5 is a perspective view illustrating a micro needle unit in FIGS. 1, and

FIG. 6 is a view illustrating a using state in which the micro needle stamp in FIG. 1 is coupled with a vibration supply means.

### BEST MODE

Hereinafter, a preferred embodiment according to the present invention will be described in detail with reference to attached drawings.

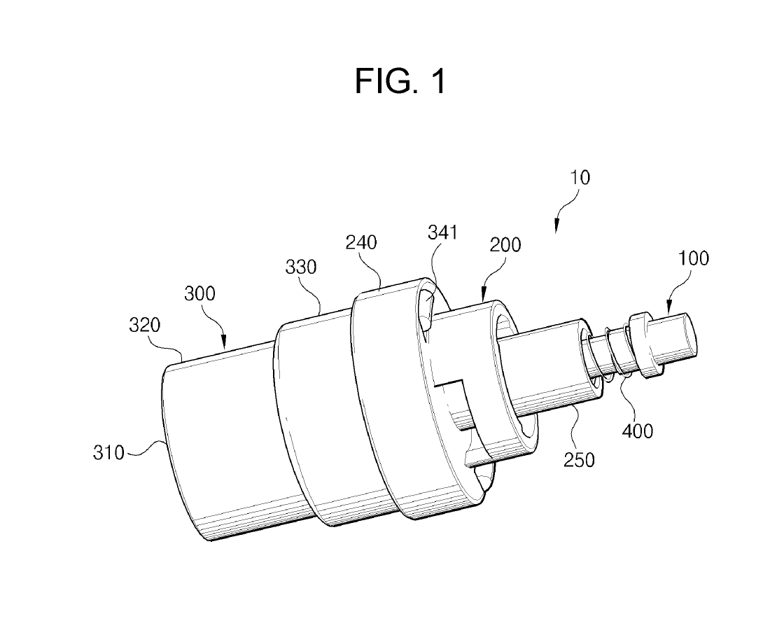

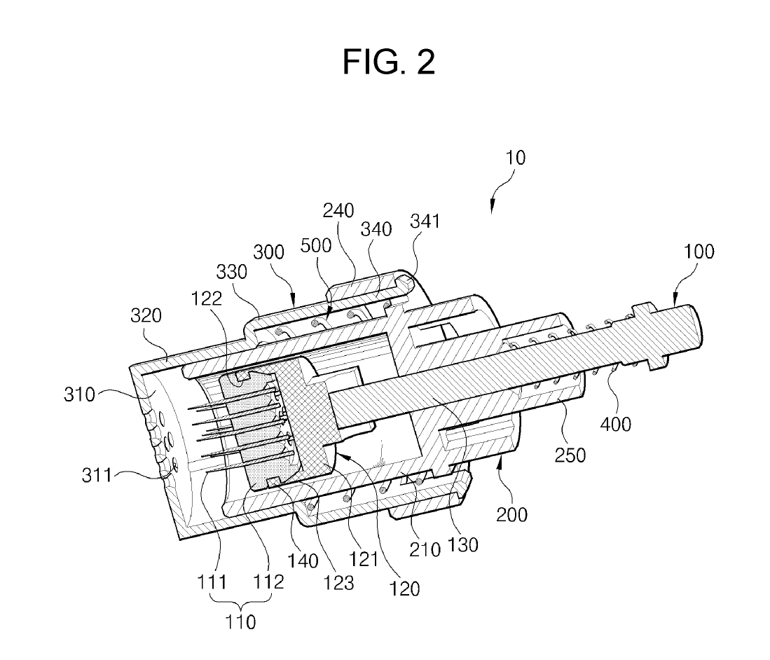

FIG. 1 is a perspective view illustrating a micro needle stamp according to an embodiment of the invention, and FIG. 2 is a sectional perspective view illustrating a micro needle stamp in FIG. 1.

With reference to FIG. 1 and FIG. 2, the micro needle stamp 10 according to an embodiment of the present invention perforates and stimulates the skin or is capable of being injected medication or the like through the opening perforated in the skin. To this end, the micro needle stamp 10 includes a micro needle unit 100, a main cover 200, an auxiliary cover 300, a first resilient body 400, and a second resilient body 500.

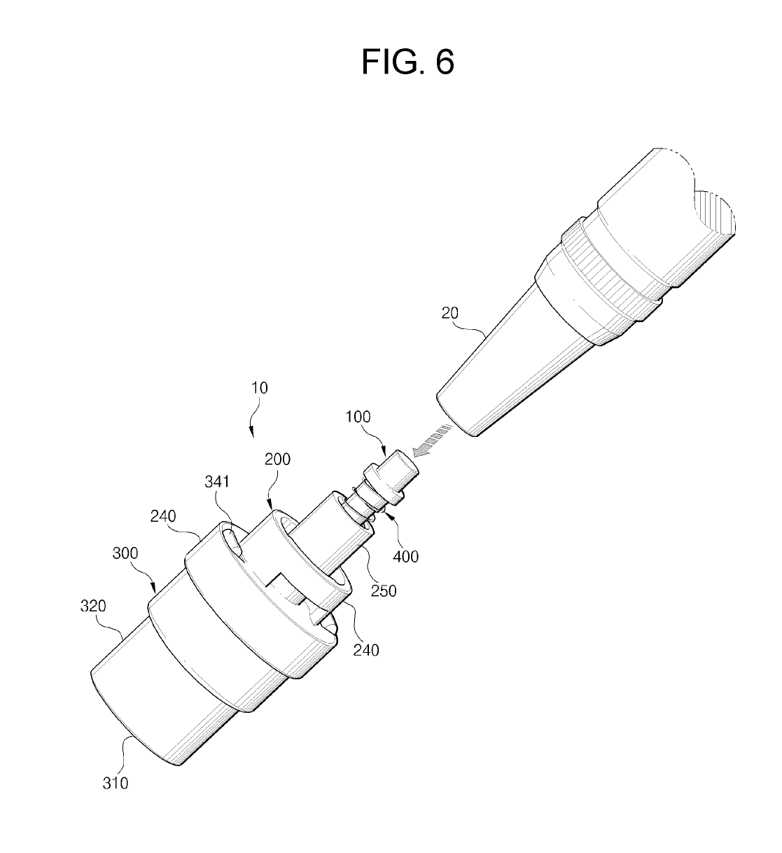

The micro needle unit 100 vibrates in the forward and backward direction by coupling with vibration supply means 20 illustrated in FIG. 6 at the backward end portion thereof. To this end, the micro needle unit 100 may be include a plurality of micro needles 111, a needle support body 110, a press block 120, and a press portion 130. Meanwhile, the vibration supply means 20 includes a motor or the like for generating vibration. However, the vibration supply means 20 has a less direct relevance to the subject matter of the present invention. Accordingly, the detailed description of the vibration supply means will be omitted.

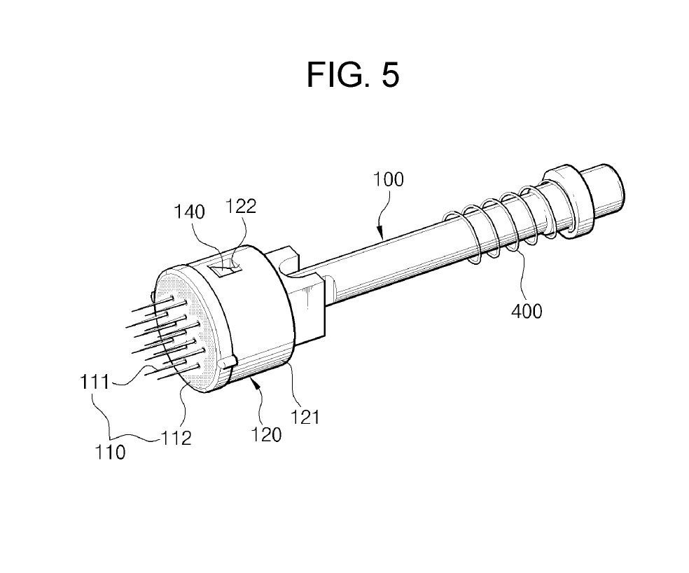

With reference to FIG. 2 and FIG. 5, the plurality of the micro needles 111 are planted in the needle support body 110 with the plurality of the micro needles 111 being exposed from the needle support body 110. The press block 120 is detachably coupled to a backward surface of the needle support body 110.

For detachably coupling the needle support body 110 and the press block 120, the needle support body 110 may be include a body 112 in which the plurality of the micro needles 111 are planted, and one projection 140 or a plurality of projections 140 that are projected from a outer surface of the body 112. According to this, the press block 120 may be include a block 121 that is in contact with a lower surface of the body 112, and a coupling portion 123 that extends from the block 121 in order to receive the lower portion of the body 112. At this time, a projection groove 122 penetrated corresponding to the projection 140 of the press block 120 is formed on the coupling portion 123. Accordingly, the needle support body 110 and the press block 120 is capable of being detachably coupled to each other by the projection 140 being fitted to the projection groove 122. Since the needle support body 110 and the press block 120 is detachably coupled to each other, in case of where the micro needle 111 is necessary to be replaced, only the needle support body 112 in which the micro needle 111 is implanted can be replaced and thus the inconvenience that replaces the entire micro needle unit 100 is eliminated and the cost according to the replacement is reduced.

The press portion 130 is connected to the backward surface of the press block 120. As illustrated in FIG. 6, the vibration supply means 20 is coupled to the press portion 130 and thus the skin is repeatedly treated by using the vibration supplied in the forward and backward direction.

The main cover 200 receives the micro needle unit 100 and slidably supports the micro needle unit 100. In the accompanying drawings, the main cover 200 is shown in the a cylindrical shape. However, the shape of the main cover is not limited to the cylindrical shape. For example, when the main cover 200 may be combined with the auxiliary cover 300 to be described later and thus may slide, it will be possible to use various shapes of embodiments, such as a tetragon.

The main cover 200 may be include a receiving portion 210, a support portion 220, a connection portion 230, an extension portion 240, and an expansion portion 50, as illustrated in FIGS. 1 and 2. A receiving space 210 that receive the needle support body 110 and the press block 120 is formed in the receiving portion 210. The support portion 220 slidably supports the press portion 130. The connection portion 230 integrally connects the receiving potion 210 and the support portion 220 to each other. The extension portion 240 has a cylindrical shape and integrally extend from the connection portion 230 to be spaced from the outside surface of the receiving portion 210. The expansion portion 250 is expanded from the support portion 220 to form a separation space between the press portion 130 and the expansion portion 250.

The plurality of the openings 311 are formed in the auxiliary cover 300 to correspond to the plurality of the micro needles 111. Further, the auxiliary cover 300 restricts the main cover 200 from relatively moving in the backward direction and is slidably coupled with the outer surface of the main cover 200. To this end, the seconary cover 300 includes a front surface portion 310, a first sliding portion 320, a second sliding portion 330, and restricting means.

The front surface portion 310 is disposed on the front side of the needle support body 110 with a gap and a plurality of openings 311 are formed on the front surface portion 310 corresponding to the plurality of the micro needles 111. The first sliding portion 320 extends from the front surface portion 310 and is slidably in face-contact with the outer surface of the receiving portion 210. In addition, the second sliding portion 330 is integrally expanded from the first sliding portion 320. Accordingly, the second sliding poriton 330 is disposed from the outer surface of the receiving portion 210 to the outside with a gap and is slidably in face-contact with the inside surface of the extending portion 240. Meanwhile, the restricting means restricts the extension portion 240 from moving to the backward side. To this end, one projection 340 or a plurality of projections 340 extended from the second sliding portion 330 to the backward side as in the drawings is provided as the restricting means. The retaining projection 341a is provided in an end portion of the projection portion 340. At this time, a retaining groove 241 is penetrated corresponding to the retaining projection 341a in the extension portion 240. According to this, the movement of the extension portion 240 in the backward direction by the retaining projection 341a is restricted and thus the movement of the main cover 200 including the extension portion 210, the support portion 220, the connection portion 230, the extension portion 240 and the expansion portion 250 integrally extended with the extension portion 240 in the backward direction is restricted.

The first resilient body 400 is interposed between the micro needle unit 100 and the main cover 200 and may be interposed between the press portion 130 and the expansion portion 250. The first resilient body 400 provides a resilient restoring force, when the micro needle unit 100 moves relative to the main cover 200. More specifically, when the press portion 130 moves in the forward direction and moves relative to the expansion portion 250, the resilient restoring force that causes the press portion to be restore an original position is provided.

The second resilient body 500 is interposed between the main cover 200 and the auxiliary cover 300, and may be interposed between the extension portion 240 and the second sliding portion 330 as illustrated in the drawings. The second resilient body 500 provides the resilient restoring force when the main cover 200 moves relative to the auxiliary cover 300. More specifically, the resilient restoring force that restores to an original position is provided when the extension portion 240 moves relative to the second sliding portion 330 by the extension portion 240 moving in the forward direction by the user.

In the micro needle stamp 10 according to the present invention as described above, when the micro needle 111 moves from one portion of the skin to the other portion in the lateral direction, the operation thereof will be performed as following procedure.

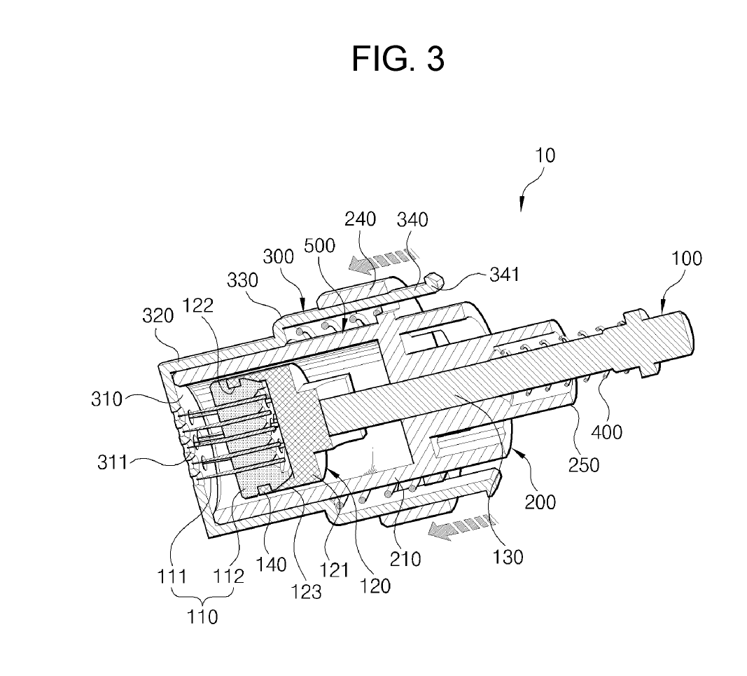

First, in a case where the treatment to one portion of the skin is requiried, as illustrated in FIG. 3, the user moves the main cover 200 in the forward direction. To this end, the user grips at the extension portion 240 of the main cover 200 and provides force that pushes in the forward direction, to the extension portion 240 thereof. Accordingly, the micro needle 111 moves up to a front surface portion 310 of the auxiliary cover 300.

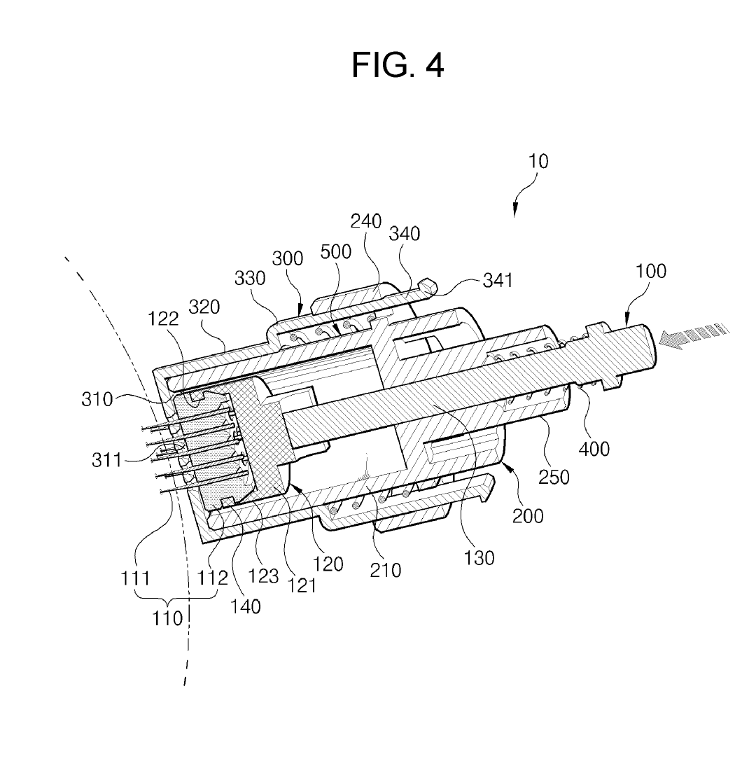

Next, as illustrated in FIG. 4, when the vibration is provided to the press portion 130 by the vibration supply means 20, the required skin treatment can perform by the micro needle 111 being drawn out through the opening 311 of the front surface portion 310.

Then, the user releases the forward movement of the main cover 200, in a case where the treatment to one portion of the skin is finished and the main cover is required to move to the other portion in the lateral direction. More specifically, the main cover 200 is returned to the original position by the resilient restoring force of the second resilient body 500 as in FIG. 2, by the user releasing the force that causes the extension portion 240 of main cover 200 to move in the forward direction. Accordingly, the micro needle 111 is introduced into an inside of a front surface portion 310 of the auxiliary cover 300.

Accordingly, it is fundamentally prevented the skin from being damaged according to movement of the micro needle 111 in the lateral direction. In particular, the continuity of the treatment operation can be maintained since user can perform the treatment by moving the main cover 200 in the forward direction, at the same time moving the micro needle 111 in the lateral direction.

While the present invention has been described with respect to the specific embodiments illustrated in drawings, this is merely exemplary. Further, it will be apparent to those skilled in the art that various changes and modifications may be made without departing from the spirit and scope of the invention as defined in the following claims. Therefore, the true technical protection scope of the invention should be defined by the appended claims.

1. A micro needle stamp, comprising:
a micro needle unit in which a plurality of micro needles are planted;
a main cover in which the micro needle unit is received and which supports to be slidable the micro needle unit;
an auxiliary cover on which a plurality of openings are formed to correspond to the plurality of the micro needles, which restricts the main cover from relatively moving in the backward direction and couples with an outer surface of the main cover in a slidable manner;
a first resilient body that is interposed between the micro needle unit and the main cover; and
a second resilient body that is interposed between the main cover and the auxiliary cover.

2. The micro needle stamp according to claim 1,
the micro needle unit includes:
the plurality of the micro needles;
a needle support body in which the plurality of the micro needles are panted to be exposed in the forward direction;
a press block that detachably couples to a backward surface of the needle support body; and
a press portion that connected to a backward surface of the press block.

3. The micro needle stamp according to claim 2,
the needle support body includes a body in which the plurality micro needles are planted; one projection or a plurality of projections projected from a outer surface of the body, and
wherein the press block includes a block that is in contact with a bottom surface of the body; and a coupling portion which extends from the block to receive the bottom portion of the body and has a projection groove penetrated corresponding to the projection.

4. The micro needle stamp according to claim 2,
the main cover includes:
a receiving portion that has a receiving space receiving the needle support body and the press block;
a support portion that slidably supports the press portion;
a connection portion that integrally connects the receiving portion with the support portion;
a extension portion that integrally extends from the connection portion to be apart from a outer surface of the receiving portion, and
an expansion portion that expands from the support portion to have a separation space between the press portion and the support portion.

5. The micro needle stamp according to claim 4,
the first resilient body is interposed between the press portion and the expansion portion.

6. The micro needle stamp according to claim 4,
the auxiliary cover includes:
a front surface portion that is spaced in front of the needle support body and has a plurality of openings corresponding to the plurality of the micro needles;
a first sliding portion that extends from the front surface portion and is slidably in face-contact with an outer surface of the receiving portion;
a second sliding portion that integrally extends to be expanded from the first sliding portion and is spaced from the outer surface of the receiving portion to the outside and is slidably in face-contact with an inner surface of the extension portion; and
restricting means that restricts the extension portion from moving in the backward direction.

7. The micro needle stamp according to claim 6,
wherein the restricting means is one projection portion or a plurality of projection portions that extend from the second sliding portion to the backward side and has a retaining projection at the end thereof, and a retaining groove corresponding to the retaining projection is penetrated through the extension portion.

8. The micro needle stamp according to claim 6,
wherein the second resilient body is interposed between the extension portion and the second sliding portion.
